(19) United States
(12) Patent Application Publication
(10) Pub. No.: US 2022/0339217 A1
(43) Pub. Date: Oct. 27, 2022

(54) PROBIOTIC FORMULATIONS FOR LIVER AND ORAL HEALTH AND ENHANCEMENT OF IMMUNITY

(71) Applicant: S. A. PHARMACHEM PRIVATE LIMITED, Mumbai (IN)

(72) Inventors: Sagar D. MANDAWGADE, Mumbai (IN); Anil K Jain, Mumbai (IN); Ratnesh D. Jain, Mumbai (IN); Devashre N Jahagirdar, Pune (IN)

(21) Appl. No.: 17/788,490

(22) PCT Filed: Dec. 21, 2020
(86) PCT No.: PCT/IB2020/062268

§ 371 Date: Jun. 23, 2022

(30) Foreign Application Priority Data
Dec. 24, 2019 (IN) 201921053624
Feb. 15, 2020 (IN) 201921046562

(51) Int. Cl. A61K 35/745; A61K 35/747; A61P 1/16

(52) U.S. Cl. CPC A61K 35/745; A61K 35/747; A61P 1/16

(57) ABSTRACT

The present invention provides novel probiotic formulation for Liver health and enhancing immunity comprising a combination of specific probiotic bacterial strains belonging to the genera Lactobacillus and Bifidobacterium. The invention also provides a probiotic formulation for oral health. The said probiotic formulations are unique in being a low water activity blend and consist of oro-dispersible properties.

### CROSS-REFERENCE TO RELATED APPLICATIONS

This application is a National Stage entry of International Patent Application PCT/IB2020/062268, filed 21 Dec. 2020, published as WO 2021/130647 A1 and titled PROBIOTIC FORMULATIONS FOR LIVER AND ORAL HEALTH AND ENHANCEMENT OF IMMUNITY, claims the benefit of and priority to Indian Provisional Application 201921046562 filed Feb. 15, 2020 and Indian Provisional application 201921053624, filed Dec. 24, 2019, each of which is incorporated herein by reference in its entirety.

### FIELD OF INVENTION

The present invention relates generally to probiotic formulation for liver health and enhancing immunity comprising a combination of specific probiotic bacterial strains belonging to the genera Lactobacillus and Bifidobacterium. The invention also provides a probiotic formulation for oral health. The said probiotic formulation is unique in being a low water activity blend and consists of oro-dispersible properties.

### BACKGROUND

Humans coexist with an enormous quantity of microbial organisms collectively termed microbiota. Human gastrointestinal tract is the most heavily colonized site, and the colon contains more than two-thirds of the microbial load. Overall, our gut has approximately 100 trillion (10¹⁴) microbes, which make up approximately 1 to 2 kilograms of our weight. (Physiological reviews, 2019, 90:859-904). The number of microbial species estimated to exist in a human gut is more than 1800. In the human gut the bacterial density gradient progressively increases from the stomach to the colon. The vastness of the human microbiota is evident given that the bacterial cells in the human gut outnumber human cells by a factor of 10 and microbial genes outnumber human genes by a factor of 100. There are variations in the predominant bacterial species not only along the length of the gastrointestinal tract but also from the lumen to the epithelium. Gut microbiota perform diverse immunologic, digestive, and metabolic functions. They can produce energy by means of specialized digestion of complex polysaccharides that cannot otherwise be digested by humans. Colonic microbes can produce short-chain fatty acids like acetate, butyrate, and propionate by metabolizing these polysaccharides (Proceedings of the Nutrition Society, 2003, 62:67-72).

The close interaction of the gastrointestinal tract and the liver and the fact that the nutrients absorbed by the gut first reach the liver have fostered use of the term gut-liver axis. Cirrhosis patients have colonic microbiota that are different from that of healthy control subjects (Current microbiology, 2012, 65: 7-13). Reports indicate the progressive decrease in the ratio of Bifidobacterium to Enterobacteriaceae accompanying progression of liver disease in a range of subjects, from healthy controls to subjects with decompensated hepatitis B virus cirrhosis to asymptomatic carriers and subjects with chronic hepatitis B. This indicates that changes in gut microbiota seem to mirror changes in severity of disease (Microbial ecology, 2011, 61: 693-703). Changes in microbiota have also been reported in non-alcoholic fatty liver disease (NAFLD), hepatic encephalopathy, alcohol-related liver disease, and hepatocellular carcinoma. Gut microbiota may cause NAFLD by luminal ethanol production, causing a leaky gut and metabolic endotoxemia, or by metabolizing choline, which is no longer available for the liver (Nutrition, Metabolism and Cardiovascular Diseases, 2012, 22:471-476).

The present invention provides a probiotic composition formulated in reducing the pro-inflammatory cytokines that play a major role in aggravating diseases like non-alcoholic steatohepatitis, NAFLD, cirrhosis, etc. The invention also provides reduction of the hepatocellular stress by reducing the levels of lipids accumulated in the hepatic cells, ALT (alanine aminotransferases) and reactive oxygen species generated.

Furthermore, several beneficial effects of probiotics on the host intestinal mucosal defences system have been identified. These include blocking pathogenic bacterial effects by producing bactericidal substances and competing with pathogens and toxins for adherence to the intestinal epithelium. For intestinal epithelial homeostasis, probiotics promote intestinal epithelial cell survival, enhance barrier function, and stimulate protective responses from intestinal epithelial cells. Most importantly, modulation of the immune system is one of the most plausible mechanisms underlying the beneficial effects of probiotics on human health. Probiotics have been found to enhance the innate immunity and modulate pathogen-induced inflammation via toll-like receptor-regulated signalling pathways (Current opinion in gastroenterology, 2011. 27:496-501). Probiotic-derived factors mediate probiotic action in the regulation of host immune responses. Probiotics exert different levels of immune-regulatory effects in a host-dependent manner, including gene expression, protein synthesis, signalling pathways in immune cells and in intestinal epithelial cells. The invention disclosed herein also comprises a probiotic composition formulated to enhance immune health.

Over the past decade, accumulating evidence has linked probiotics to better dental health outcomes and reducing the incidence of oral diseases such as halitosis, periodontitis, and oral candidiasis (Archives of Oral Biology, 2017, 83:187-92). The most commonly used probiotic bacterial strains belong to the genera Lactobacillus and Bifidobacterium (Current opinion in biotechnology, 2005, 16:204-211). These bacterial genera are regarded as a part of the normal human microbiota. In the oral cavity, lactobacilli usually comprise fewer than 1% of the total cultivable microbiota, but no species specific to the oral cavity has been found. In contrast, some species are found in both oral and fecal samples (Journal of medical microbiology, 2008, 57: 1560-1568). Species commonly isolated from saliva samples include L. paracasei, L. plantarum, L. rhamnosus, and L. salivarius. Culture-based studies suggest that bifidobacteria are among the first anaerobes in the oral cavity. (8) Indeed, both lactobacilli and bifidobacteria can be found in breast milk, suggesting early exposure of the oral cavity to these bacteria (Journal of pediatric gastroenterology and nutrition, 2009, 49: 349-354.). Bifidobacterial species isolated from oral samples include B. bifidum, B. dentium, and B. longum (Appl. Environ. Microbiol., 2008, 74: 6457-6460).

Lactobacilli and bifidobacteria are generally regarded as safe, and, since the early writing of Metchnikoff, even more fermented food products have been associated with health benefits. In respect to normal microbiota and oral health, there seem to be differences in the ability of lactobacilli isolated from caries-active or healthy subjects to inhibit Streptococcus mutans in vitro (European journal of oral sciences, 2007, 115:308-314). In addition, the species composition of both Lactobacillus and Bifidobacterium microbiota is different between patients with periodontitis and those who are periodontally healthy (Bioscience, biotechnology, and biochemistry, 2007, 71(1), 152-157). On the other hand, both lactobacilli and bifidobacteria are also associated with dental caries (Journal of clinical microbiology, 2008, 46: 1407-1417). In addition, caries-associated lactobacilli and bifidobacteria have been characterized as exogenous and opportunistic colonizers possibly acquired from food (Caries research, 2007 41:2-8).

While the effects of many lactobacilli or bifidobacteria have been studied for oral health, the specific effect of the combination has not been elucidated. The present invention provides a combination of lactobacilli and bifidobacteria in maintaining and/or improving oral health.

### BRIEF DESCRIPTION OF DRAWINGS

FIG. 1. Viability of probiotic bacteria in Mammalian Culture conditions (Liver).

FIGS. 2a, 2b, 2c, 2d, 2e, 2f, and 2g. Pro-inflammatory Cytokine Assay (Liver): 2a) IL-1β, 2b) IL-8, 2c) IL-10, 2d) IL-12, 2e) IL-17A, 2f) IFN-γ, 2g) TNF-α.

FIG. 3. Lipid Accumulation Assay.

FIG. 4. Intracellular ALT level colorimetry.

FIG. 5. Reactive Oxygen species in different experimental groups.

FIG. 6. Results of Cell Proliferation Assay using probiotic combination for liver health

FIGS. 7a, 7b, 7c, 7d, and 7e. Viability of probiotic bacteria in Mammalian Culture conditions (Immune system): 7a) L. rhamnosus, 7b) L. acidophilus, 7c) L. casei, 7d) L. plantarum, 7e) B. breve.

FIG. 8. Pro-inflammatory Cytokine Assay (Immune system).

FIG. 9. Results of Proliferation assay using probiotic combination for enhancing the immune system.

FIG. 10. Effect of supplements on viability.

FIG. 11. Viability of probiotic bacteria in Mammalian Culture conditions (Oral).

FIGS. 12a, 12b, 12c, and 12d. Results of pro-inflammatory cytokine assay using the probiotic combination for oral health. 12a) IL-6, 12b) IL-8, 12c) IL-1β, 12d) TNF-α.

FIG. 13. Results of Proliferation assay using probiotic combination for oral health.

### DETAILED DESCRIPTION OF THE INVENTION

Probiotic Composition for Liver Health

The probiotic composition of the present invention for improving liver health comprises 0.5-1.5×10⁹ CFU of L. plantarum, 0.5-1.5×10⁹ CFU of L. paracasei, 0.5-1.5×10⁹ CFU of L. acidophilus, 0.5-1.5×10⁹ CFU of B. Lactis, 0.5-1.5×10⁹ CFU of B. breve.

The said composition of the invention is formulated by the process as described below:
a. making a blend of inert excipients selected from the group consisting of microcrystalline cellulose, maltodextrin, lactose, starch, FOS, Alpha-GOS, magnesium stearate and silicon dioxide;
b. thawing and mixing the probiotic cultures with suitable portion of blend from (a);
c. mixing blend of (b) with that of step (a) under controlled processing parameters wherein the said parameters comprise temperature of 18 to 20° C. and relative humidity of 20 to 25%;
d. adding and mixing non-probiotic components selected from anticaking and lubricating agents in blend of (c); and
e. optionally adding and mixing flavours to the blend of (d).

The above process is specific and unique in that as the formulation generated is a low water activity blend necessary for maintaining the shelf-life and has properties of oro-dispersible powder. The shelf-life as meant here is about at least 24 to 36 months.

The inert excipients as per the invention include but not limited to microcrystalline cellulose, maltodextrin, lactose, starch, FOS, Alpha-GOS, magnesium stearate, silicon dioxide and the likes thereof. The anticaking agent herein is silicon dioxide or magnesium stearate and lubricating agent is magnesium stearate.

The formulation of the invention is used for maintaining sound liver health. By liver health, it is meant that the formulation is used for the prevention and/or treatment of liver including but not limited to cirrhosis, Non-alcoholic fatty liver disease (NAFLD), Non-alcoholic steatohepatitis (NASH), alcoholic liver disease, and detoxification of liver.

In an important embodiment, the formulation the probiotic composition of the invention is formulated as capsules, sachets, stick pack, reconstitutes would be the dosage forms that will contain appropriate powder type such as—plain, granular, mouth melt, oro-dispersible powder, all of which are equally effective for the said use of maintaining the liver health.

In one embodiment, the probiotic formulation significantly reduces alanine amino transferase activity (ALT enzyme).

In the prior art, maximum reported range for probiotic dosage in human adults is 1-10 billion/day. The study refers and compares to the highest reported dosage values. However, considering the efficacy of the formulation in the present invention, synergistic effect of the probiotic composition is achieved within the recommended dosage range (5 billion CFU/day).

The combined probiotics of the invention enhances human immunity and improves gastrointestinal functions via reducing inflammation targeting cytokines and has anti-oxidization functions. Additionally, the strain selection helps in specifically targeting the free radical generation and helps in eliminating the harmful oxidants. Furthermore, it also reduces the levels of pro-inflammatory cytokines.

The probiotic liver composition acts synergistically with respect to the decrease in the expressed cytokine level. The synergism can be seen as reduction in the levels of cytokines. The values obtained from the tests performed justify the synergism and not an additive effect.

Probiotic Formulation for Enhancing Immune Health

The probiotic composition of the present invention for enhancing immune health comprises 1-2×10⁹ CFU of B. breve, 1-2×10⁹ CFU of L. casei, 1-2×10⁹ CFU of L. rhamnosus, 1-2×10⁹ CFU of L. acidophilus, and 1-2×10⁹ CFU of L. plantarum and vitamins, preferably vitamin B12 and minerals like nucleotides, CaCl₂, B12, Zn, Mg. The recommended dose for improving the immune health is 10 billion CFU/day that is proved to be significant in reducing the primary pro-inflammatory cytokine (IFN-γ) responsible in triggering inflammatory cascade. Supplements added here play a role in maintenance of the probiotic compositions and also serve as immune boosters in conditions such as Rheumatoid arthritis, auto-immune disorders and multiple sclerosis.

The above composition is formulated in a similar fashion as that of the probiotic composition for liver health as provided above.

The probiotic formulation of the invention is useful in improving or enhancing immune health in general and is used to reduce the deleterious effects of disorders including but not limited to inflammatory bowel syndrome (IBS), rheumatoid arthritis, auto-immune disorders, and multiple sclerosis.

The probiotic formulation of the present invention with added supplements is shown to have increased activity in terms of decrease in the pro-inflammatory cytokine condition.

Probiotic Formulation for Improving Oral Health

The present invention also provides a combination of Lactobacillus and Bifidobacterium for improving the oral health. The probiotic combination of the invention contains 1 to 2×10⁹ CFU of L. salivarius, 0.5 to 1×10⁹ CFU of L. rhamnosus, 0.5 to 1×10⁹ CFU of L. plantarum, 0.5 to 1×10⁹ CFU of B. lactis, and 1 to 2×10⁹ CFU of B. breve, with a total of 4 billion CFU/day. The findings corroborate with the reported dosage value resulting in attaining significant reduction in the inflammatory signals.

In one embodiment, the probiotic combination of the present invention encourages the growth of probiotic bacteria, which in turn inhibits the growth of more harmful bacteria, genera in the oral cavity and the distal gastrointestinal tract (Candida albicans). The combination is also effective in the degradation of toxins generated by pathogenic bacteria (S. mutans or C albicans). These pathogens mentioned carry high burden of oral infections and reports mention the use of antibiotics for the therapy. Use of the probiotic combination will lessen the antibiotic dosage as well as control the infection by decreasing the infection. The claim suggests the reduction of the potent oral infection.

In another embodiment, the probiotic combination of the present invention facilitates the formation of aggregates and adherence on oral tissues. This aspect of the combination is unique as the strains used display aggregation property which leads to prolonged adherence on the oral tissues. In the presence of supplements like simple and complex sugars and also globular proteins that participate in increased adherence, the probiotic combination will enhance the adherence efficiency.

The oral composition of the invention contains Bifidobacterium sp. and Lactobacillus sp., thereby normalizing the acid production by Lactobacillus species. This will assist in the regulation of oral pH. The use of Lactobacillus sp. lowers the pH resulting in acidic condition in oral cavity. Use of Bifidobacterium sp. helps in maintaining and regulating the pH in the oral cavity.

The concentration of Lactobacillus sp. facilitates in the maintenance of the gut microflora thereby contributing towards the enhanced immunity.

The proposed bacterial load in the combination is useful for controlled release of the bacteria when entrapped in a film or chewable gum. Also, the effectiveness of the adhered bacteria of the stated count can effectively reduce the inflammation. The bacterial load claimed is more than that mentioned in the probiotics which are ingested orally as suspension.

The probiotic composition of the invention is envisaged to be immobilized/trapped in a biofilm wherein it can deliver the controlled release of the bacterial load and assist in enhancing the viability of the released bacteria at the specific time point.

The in vitro tests conducted on the mammalian cell line subjected with the oral probiotic formulation depict the survival of the bacterial load, thereby increasing the efficacy of the probiotic strains in the in vitro conditions. The cytokine quantitation of the cells in presence of stress shows a significant decrease in the level of inflammatory cytokine.

The test conditions represent the prophylactic as well as a curative composition for an immune-modulatory disease. The oral probiotic composition of the invention is useful for maintaining oral health in general as well as in diseases including but not limited to Periodontitis, Gingivitis, Halitosis, bad breath, and inflammation provoking conditions.

The probiotic formulations of the invention were tested for their efficacy to ascertain their role in liver and oral health as well as in enhancing the immunity, the details of which are provided as Examples. The Examples provided here are for better understanding and illustrative purposes only and not to be construed as limiting the scope of the invention.

### I. Examples for Formulations Improving Liver Health

Materials

a) Cell Culture

HepG2 cells were purchased from National centre for cell sciences (NCCS, Pune) and cultured in humidified atmosphere of 5% CO2, 95% air at 37° C. Briefly, HEpG2 cells were maintained in a growth medium containing the following components: Dulbecco's modified Eagle's medium (DMEM) with high glucose and 10% fetal calf serum.

b) Probiotic Composition

HepG2 cells were treated with a combination of probiotic bacterial load, each strain with a specific principle. The probiotic strains were recruited from DuPont and were already tested for strain stability. The composition contains L. plantarum (10⁹ CFU), L. paracasei (10⁹ CFU), L. acidophilus (10⁹ CFU), B. lactis (10⁹ CFU), B. breve (10⁹ CFU). Stress inducing agent was taken as LPS (Lipopolysaccharide) and test conditions were considered with exposure of LPS before and after the probiotic treatment.

### EXAMPLE 1

### Probiotic Viability in Mammalian Culture Conditions

Probiotic colony count was carried out by colony counting method, analysing the colony grown on respective agar plate after incubating for 48 hours at 37° C. (13).

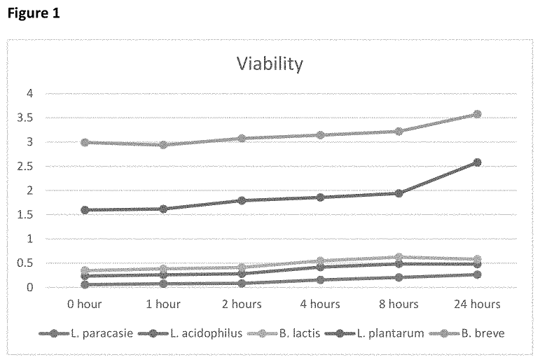

The results of individual strains for analyzing its viability depict the sustenance of the bacterial population till 8 hours. The maximum time point being 24 hours depicted decrease in the viability of strains like Lactobacillus acidophillus, Bifidobacterium breve, and Bifidobacterium lactis and increased growth in Lactobacillus plantarum and Lactobacillus paracasei. The combination of strains proves to be synergistically effective in the cumulative effect on the hepatocellular conditions (FIG. 1).

TABLE 1
Probiotic Colony Count

| Strains | Number of colonies |
|---|---|
| L. paracasei | 37 × 10⁸ |
| L. acidophilus | 29 × 10⁸ |
| L. plantarum | 4 × 10⁸ |
| B. lactis | 17 × 10⁸ |
| B. breve | 18 × 10⁸ |

The colony count shows the increase in the number of bacteria per ml of the volume plated. The number represents the number of bacteria per ml of the solution.

### EXAMPLE 2

### Cytokine Analysis

Cytokine analysis was performed using a commercial multiplex array inflammatory cytokine assay kit (Qiagen, MEH-004A). The kit was based on sandwich ELISA test. Results were analysed on a plate reader at 450 nm.

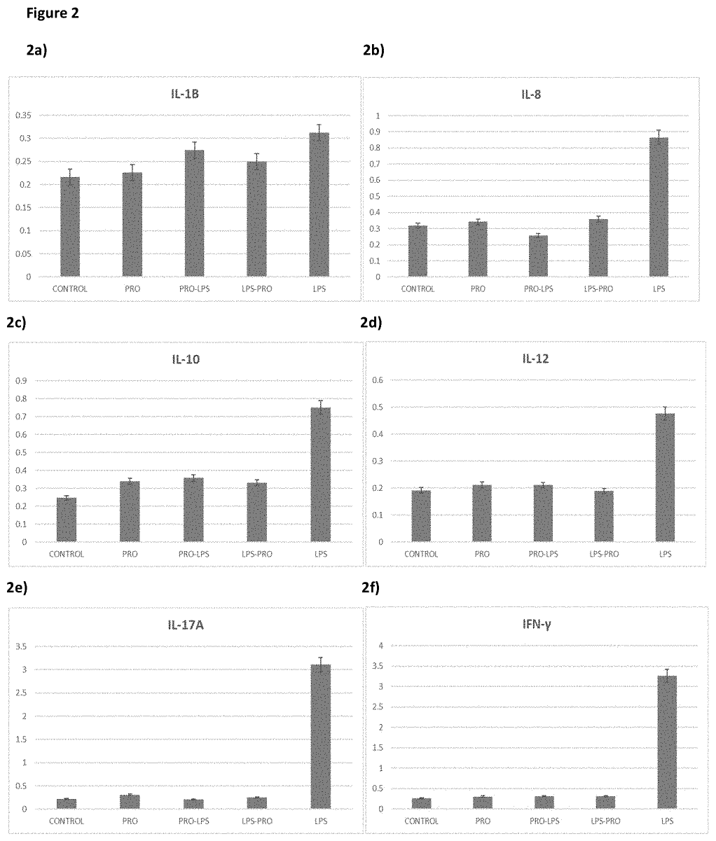

The inflammatory cytokine assay performed represents a significant decrease even in the presence of a potent inflammatory inducer LPS of cytokines including IL-1B, IL-8, IL-10, IL-12, IL-17A, IFN-γ and TNF-α. The levels of inflammatory cytokine were seen to be decreased after analysing the levels (FIGS. 2a-2g). This represents that the reduced levels are playing a role in reverting the normal tissue physiological condition, thereby, covering the treatment of various hepatocellular disease.

### EXAMPLE 3

### Lipid Accumulation

HepG2 cells were grown in a 96 well plate in the absence or presence of fatty acids were washed with PBS and then applied to Oil Red O staining assay according to the protocol of a commercial kit (Abcam, #133102). Briefly, cells were first washed with PBS and fixed with formalin solution for 15 minutes. Positive control taken for this assay was oleic acid and incubated for 4 hours. The fixed lipid droplets were then stained with Oil Red O solution for 30 minutes at room temperature. Microscope images were taken to visualize red oil droplets staining in cells treated with and without probiotic treatment.

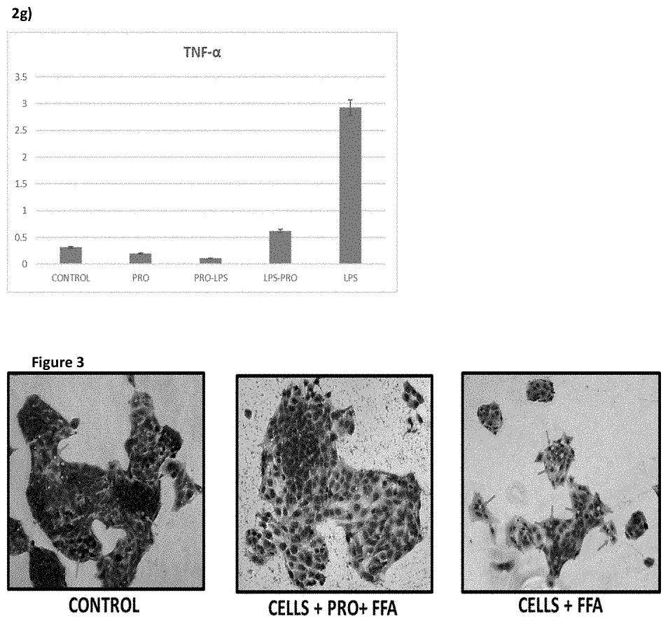

Untreated cells were shown increased levels of lipid accumulated (A), whereas when the cells were treated with probiotics it was observed that the cells had very low levels of accumulation (B) and the cells when subjected to Free Fatty Acid depicted decrease in cell number as well as significant increase in the lipid molecules in the intracellular region of the cells (FIG. 3).

### EXAMPLE 4

### ALT Enzyme Assay

Cells were cultured in 96 well plate under normal culture conditions and were incubated overnight. Respective individual and combined treatments of probiotics and stress inducer (LPS) were subjected, and the protocol was followed as per the kit. (Alanine Aminotransferase Activity Assay Kit, Sigma, MAK052).

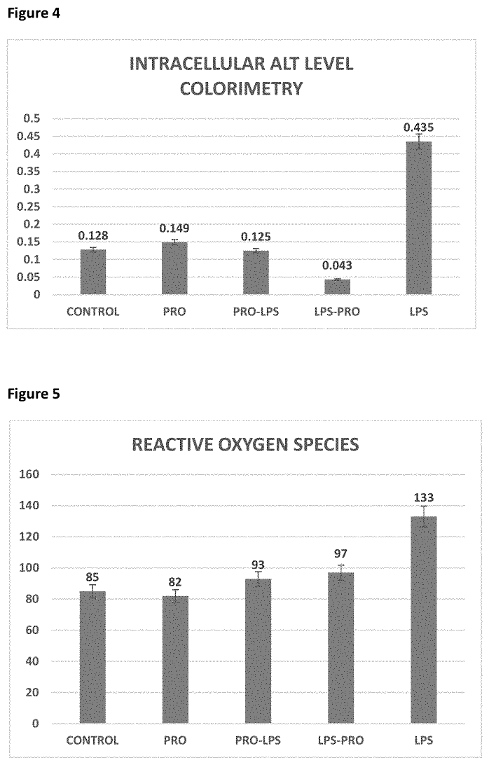

The test performed showed very high readings as compared to the standard curve due to the interference of the phenol red from the medium. The readings support that the effect of the probiotics on the cells depict reduced extracellular ALT levels as compared to the cells treated with CCl4, positive control (FIG. 4).

### EXAMPLE 5

### Reactive Oxygen Species Generation

Adherent HepG2 cells were subjected to individual and in combination with the respective treatment of probiotics and ROS inducer (TBHP) to the final concentration of 100 μM and probiotics sample was incubated for 8 hours and TBHP treated cells were incubated for 2 hours. Further the reaction master mix was added according to the documented protocol and analysed for fluorescence intensity (λex=640/λem=675 nm) (Fluorometric Intracellular ROS Kit, MAK142).

The effect of the stress inducer TBHP is prominently visible as it accounts for maximum ROS generated, whereas there is decrease in case of the cells subjected with probiotic (FIG. 5).

### EXAMPLE 6

### Cell Proliferation Assay

Plate the cells in 96 well plate with a density of 30,000 cells/well. After overnight incubation add the desired probiotic suspension and stress inducing agent and incubate for 8 hours and 2 hours respectively. Discard media from cell cultures. For adherent cells, carefully aspirate the media. Add 50 μL of serum-free media and 50 μL of MTT solution into each well. Incubate the plate at 37° C. for 3 hours. After incubation, add 150 μL of MTT solvent into each well. Wrap plate in foil and shake on an orbital shaker for 15 minutes. Occasionally, pipetting of the liquid may be required to fully dissolve the MTT formazan. Absorbance read at OD=590 nm.

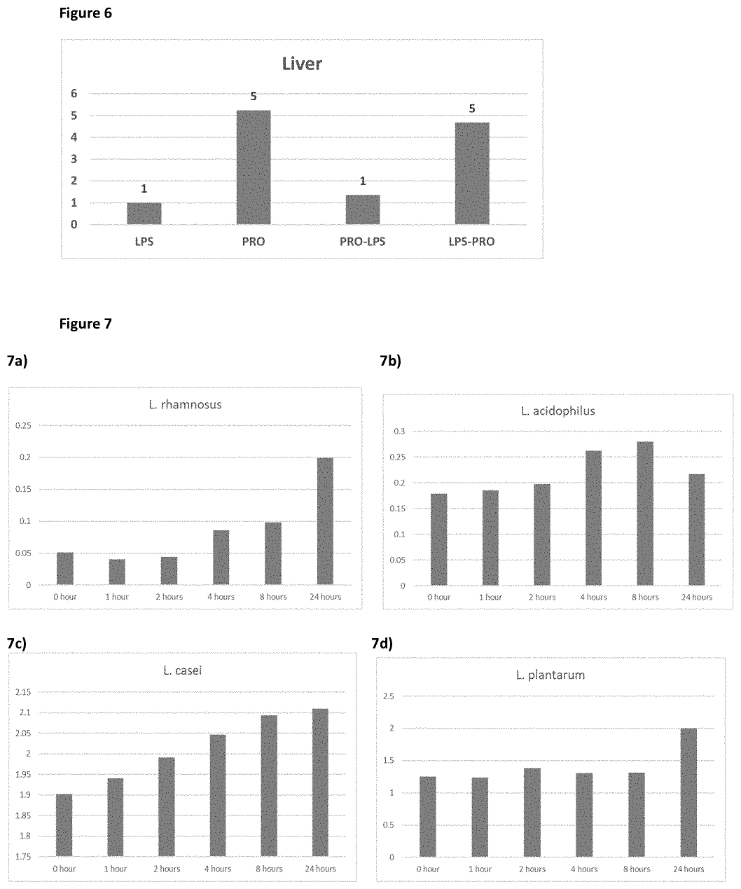

The proliferation data indicate the fold proliferation of the mammalian cells in presence of the optimized composition of the probiotic and various supplements. The graph depicts 4-fold increase in the proliferation rate of HepG2 cells when subjected to the combination of probiotic and mineral supplements. The analysis was done after inducing the stress condition to the cells with LPS, thereby supporting the results of reduction in the cellular stress biochemical compounds (FIG. 6).

### II. Examples for Formulations Enhancing Immune Health

Materials

a) Cell Culture

JAWSII cells were purchased from ATCC (American Type Cell Culture) and cultured in humidified atmosphere of 5% CO2, 95% air at 37° C. Briefly, JAWSII cells were maintained in a growth medium containing the following components: Dulbecco's modified Eagle's medium (DMEM) with high glucose and 10% fetal calf serum.

b) Probiotic Composition

JAWSII cells were treated with a combination of probiotic bacterial load, each strain with a specific principle. The probiotic strains were recruited from DuPont and were already tested for strain stability. The composition contains B. breve (10⁹ CFU), L. casei (10⁹ CFU), L. rhamnosus (10⁹ CFU), L. acidophilus (10⁹ CFU), L. plantarum (10⁹ CFU) and vitamins and minerals like Nucleotides, CaCl₂, B12, Zn, Mg. Stress inducing agent was taken as LPS (Lipopolysaccharide) and test conditions were considered with exposure of LPS before and after the probiotic treatment.

### EXAMPLE 7

### Probiotic Viability in Mammalian Culture Conditions

Probiotic colony count was carried out by colony counting method, analysing the colony grown on respective agar plate after incubating for 48 h at 37° C. (13).

The results of individual strains for analyzing its viability depict the sustenance of the bacterial population till 8 h. The maximum time point being 24 h depicted decrease in the viability of strains like Lactobacillus acidophillus and Bifidobacterium breve and increased growth in Lactobacillus plantarum, Lactobacillus rhamnosus, and Lactobacillus casei. The combination of strains proves to be synergistically effective in the cumulative effect on the immunological conditions (FIGS. 7a-7e).

The viability data obtained shows the combination of bacterial strains to be used and the duration at which the bacteria needs to be incubated with mammalian cells. The bacterial strains are chosen in a way to regulate the cumulative pH of the complete combination. Therefore, to regulate the pH drop created by the Lactobacillus spp., Bifidobacterium spp. are selected. Furthermore, the time duration optimized also supports the literature regarding the intracellular cytokine secretion. Also, further increase in the efficacy of the combination was observed upon addition of the supplements in the combination.

TABLE 2
Probiotic Colony Count

| Strains | Number of colonies |
|---|---|
| L. casei | 158 × 10⁸ |
| L. acidophilus | 29 × 10⁸ |
| L. plantarum | 4 × 10⁸ |
| L. rhamnosus | 70 × 10⁸ |
| B. breve | 8 × 10⁸ |

The colony count shows the increase in the number of bacteria per ml of the volume plated. The number represents the number of bacteria per ml of the solution.

### EXAMPLE 8

### Cytokine Analysis

The cytokine analysis was performed as provided in Example 2.

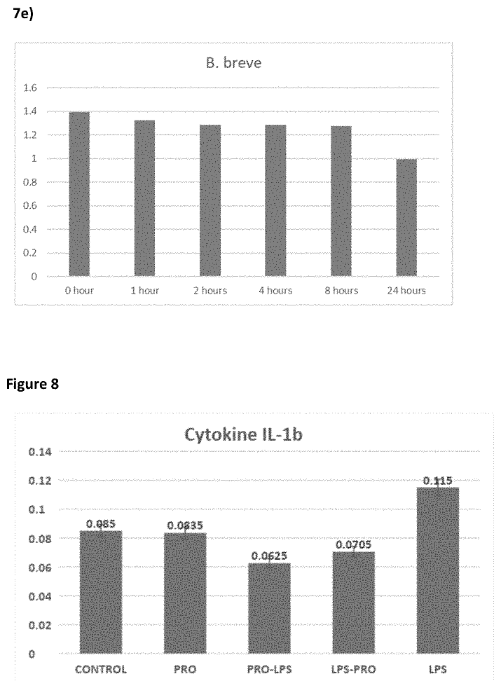

The inflammatory cytokine assay performed represents a significant decrease even in the presence of a potent inflammatory inducer LPS of cytokine IL-1B. The levels of inflammatory cytokine were seen to be decreased after analyzing the levels (FIG. 8). This represents that the reduced levels are playing a role in reverting the normal tissue physiological condition. Thereby, covering the treatment of various immune health related disease.

### EXAMPLE 9

### Cell Proliferation Assay

Proliferation assay was carried out as per the procedure provided in Example 6.

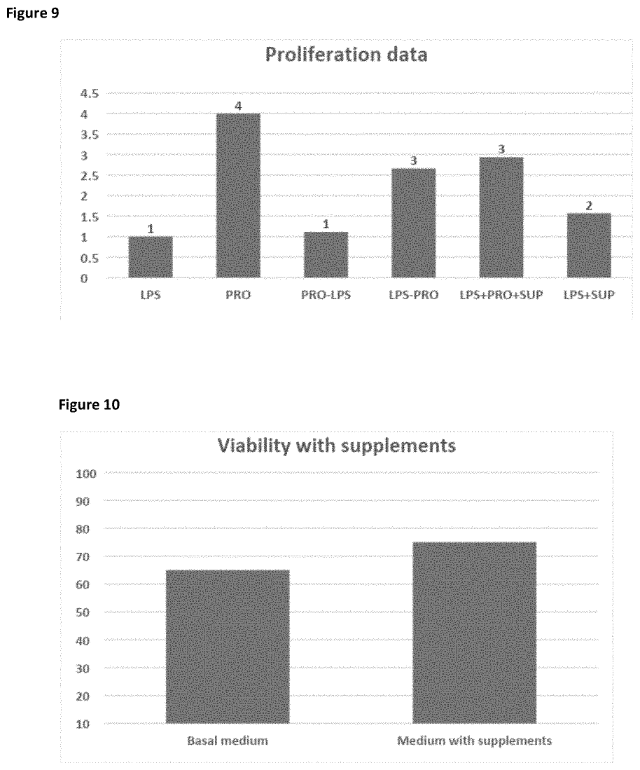

The proliferation data indicate the fold proliferation of the mammalian cells in presence of the optimized composition of the probiotic and various supplements. The graph depicts 2-fold increase in the proliferation rate of JAWSII cells when subjected to the combination of probiotic and mineral supplements (FIG. 9). The analysis was done after inducing the stress condition to the cells with LPS, thereby supporting the results of reduction in the cellular stress biochemical compounds.

The composition is ideal for suppressing the pro-inflammatory cytokine and therefore can be considered for additional treatment of inflammatory, irritable bowel disease, auto-immune conditions.

### EXAMPLE 10

### Effect of Supplements on Viability

The supplements include nucleotides (dNTPs), CaCl₂, B12, Zn and Mg, these supplements assist in the increased viability of the probiotics as well as serves as co-factors for the cellular machinery of the mammalian cells. The percent viability of the mammalian cells was observed to be increased from 68% to 75% (FIG. 10).

The mineral supplements used possess greater potential for the synergistic performance of the probiotics targeting the immune system as well as by maintaining and improving the gut microflora.

### III. Examples for Formulations Improving Oral Health

Materials

a) Cell Culture

PGF (Primary Gingival Fibroblast) cells were purchased from ATCC (American Type Cell Collection) and cultured in humidified atmosphere of 5% CO₂, 95% air at 37° C. Briefly, PGF cells were maintained in a growth medium containing the following components: Fibroblast Basal Medium [Cat no. ATCC PCS-201-030] and Fibroblast Growth kit—Low Serum [Cat no. ATCC PCS-201-041].

b) Probiotic Composition

PGF cells were treated with a combination of probiotic bacterial load, each strain with a specific principle. The probiotic strains were recruited from DuPont and were already tested for strain stability. The composition contains L. Salivarius (10⁹ CFU), L. Rhamnosus L. Rhamnosus (10⁹ CFU), L. plantarum (10⁹ CFU), B. Lactis (10⁹ CFU), B. Breve (10⁹ CFU). Stress inducing agent was taken as LPS (Lipopolysaccharide) and test conditions were considered with exposure of LPS before and after the probiotic treatment.

### EXAMPLE 11

### Probiotic Viability in Mammalian Culture Conditions

Viability of individual strains of the probiotics was evaluated by MTT that determines the absorbance based on the MTT crystal formation in the intracellular region of the mammalian cell assay (18).

The results of individual strains for analyzing its viability depict the sustenance of the bacterial population till 8 hours. The maximum time point being 24 hours depicted decrease in the viability of strains like Lactobacillus acidophillus, Bifidobacterium breve, and Bifidobacterium lacti, and increased growth in Lactobacillus plantarum and Lactobacillus paracasei (FIG. 11). The combination of strains proves to be synergistically effective in the cumulative effect on the hepatocellular conditions.

The viability data obtained shows the combination of bacterial strains to be used and the duration at which the bacteria need to be incubated with mammalian cells. The bacterial strains are chosen in a way to regulate the cumulative pH of the complete combination. Therefore, to regulate the pH drop created by the Lactobacillus spp., Bifidobacterium spp. is selected.

### EXAMPLE 12

### Probiotic Colony Count

Probiotic colon count was carried out by colony counting method, analysing the colony grown on respective agar plate after incubating for 48 hours at 37° C.

TABLE 3
Number of colonies of the probiotic strains

| Strains | Number of colonies |
|---|---|
| L. salivarius | 51 × 10⁸ |
| L. rhamnosus | 70 × 10⁸ |
| L. plantarum | 4 × 10⁸ |
| B. lactis | 17 × 10⁸ |
| B. breve | 18 × 10⁸ |

The colony count shows the increase in the number of bacteria per ml of the volume plated. The number represents the number of bacteria per ml of the solution.

### EXAMPLE 13

### Cytokine Analysis

Cytokine analysis was performed using a commercial multiplex array inflammatory cytokine assay kit (Qiagen, MEH-004A). The kit was based on sandwich ELISA test. Results were analysed on a plate reader at 450 nm.

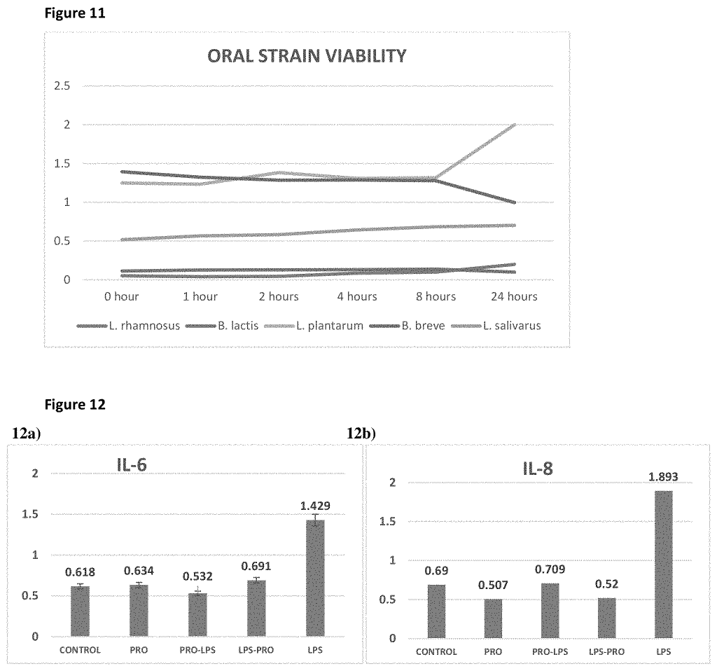

The inflammatory cytokine assay performed represents a significant decrease even in the presence of a potent inflammatory inducer LPS of cytokines including IL-1B, IL-6, IL-8 and TNF-α (FIGS. 12a-2d). Treatment conditions include a prophylactic [before the onset of disease/physiological stress] (PRO-LPS) and combination treatment given after the disease induction (LPS-PRO). The levels of inflammatory cytokine were found to be decreased after analyzing the levels. This indicates that the reduced levels are playing a role in reverting the normal tissue physiological condition thereby, establishing its use in the treatment of various oral cavity diseases.

Novel probiotic composition significantly reduces the pro-inflammatory cytokines in the presence LPS. The test conditions represent the prophylactic as well as a curative composition for an immune-modulatory disease. The oral probiotic composition covers diseases like Periodontitis, Gingivitis, Halitosis and inflammation provoking conditions.

### EXAMPLE 14

### Proliferation Assay

Cells were plated in 96 well plate with a density of 30,000 cells/well. After overnight incubation desired probiotic suspension and stress inducing agent were added and incubated for 8 hours and 2 hours respectively. Media from cell cultures were discarded. For adherent cells, the media was carefully aspirated. 50 μL of serum-free media and 50 μL of MTT solution were added to each well and the plate was incubated at 37° C. for 3 hours. After incubation, 150 μL of MTT solvent was added to each well. Plate was wrapped with foil and shaken on an orbital shaker for 15 minutes. Occasionally, pipetting of the liquid was required to fully dissolve the MTT formazan. Absorbance was read at OD=590 nm.

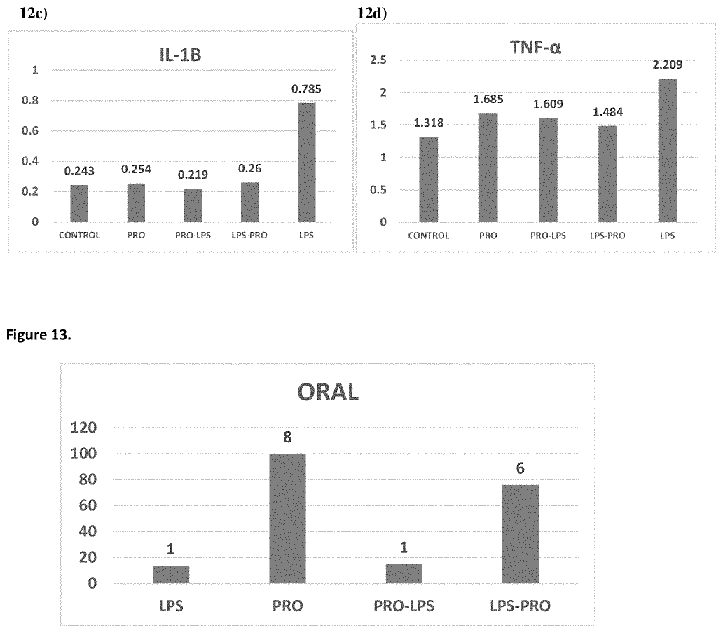

The proliferation data indicate the fold proliferation of the mammalian cells in presence of the optimized composition of the probiotic and various supplements. FIG. 13 shows 5-fold increase in the proliferation rate of PGF cells when subjected to the combination of probiotic and mineral supplements. The analysis was done after inducing the stress condition to the cells with LPS, thereby supporting the results of reduction in the cellular stress biochemical compounds.

The composition is ideal for suppressing the pro-inflammatory cytokine and therefore can be considered for additional treatment of inflammatory, gingivitis, bad breath, halitosis, periodontitis conditions.

1.-13. (canceled)

14. A probiotic formulation comprising a combination of Lactobacillus plantarum (L. plantarum), L. paracasei, L. acidophilus, Bifidobacterium lactis (B. lactis), and B. breve.

15. The probiotic formulation of claim 14, wherein the probiotic formulation is a low water activity blend.

16. The probiotic formulation of claim 15, wherein the probiotic formulation with low water activity blend consists of oro-dispersible properties and maintains shelf-life of 24 to 36 months.

17. The probiotic formulation of claim 1, wherein the combination comprises 0.5-1.5×10⁹ CFU of L. plantarum, 0.5-1.5×10⁹ CFU of L. paracasei, 0.5-1.5×10⁹ CFU of L. acidophilus, 0.5-1.5×10⁹ CFU of B. lactis, and 0.5-1.5×10⁹ CFU of B. breve.

18. A method for improving liver health or enhancing immune health in a subject in need thereof, the method comprising administering the probiotic formulation of claim 14.

19. The method of claim 18, wherein liver health refers to Non-alcoholic fatty liver disease (NAFLD), Non-alcoholic steatohepatitis (NASH), alcoholic liver disease, or detoxification of liver.

20. The method of improving liver health claim 18, wherein the combination comprises 0.5-1.5×10⁹ CFU of L. plantarum, 0.5-1.5×10⁹ CFU of L. paracasei, 0.5-1.5×10⁹ CFU of L. acidophilus, 0.5-1.5×10⁹ CFU of B. lactis, and 0.5-1.5×10⁹ CFU of B. breve.

21. The method of improving liver health claim 18, wherein administering the probiotic formulation comprising administering to the subject 5 billion CFU/day of a synergistic combination of L. plantarum, L. paracasei, L. acidophilus, B. lactis, and B. breve.

22. The method of enhancing immune health of claim 18, wherein the probiotic formulation further comprises a vitamin and a mineral, wherein the mineral is selected from the group consisting of nucleotides, CaCl₂, Zn, and Mg, and the vitamin is vitamin B12.

23. The method of enhancing immune health of claim 18, wherein the combination comprises 1-2×10⁹ CFU of B. breve, 1-2×10⁹ CFU of L. casei, 1-2×10⁹ CFU of L. rhamnosus, 1-2×10⁹ CFU of L. acidophilus, and 1-2×10⁹ CFU of L. plantarum.

24. The method of enhancing immune health of claim 18, wherein said immune health refers to inflammatory bowel syndrome (IBS), rheumatoid arthritis, an auto-immune disorder, or multiple sclerosis.

25. A probiotic formulation for improving oral health comprising a combination of Lactobacillus and Bifidobacterium, wherein the Lactobacillus is selected from the group consisting of L. salivarius, L. rhamnosus, and L. plantarum, and the Bifidobacterium is selected from the group consisting of B. lactis and B. breve.

26. The method of claim 25, wherein the combination Lactobacillus and Bifidobacterium comprises 1 to 2×10⁹ CFU of L. salivarius, 0.5 to 1×10⁹ CFU of L. rhamnosus, 0.5 to 1×10⁹ CFU of L. plantarum, 0.5 to 1×10⁹ CFU of B. lactis, and 1 to 2×10⁹ CFU of B. breve.

27. The method of claim 25, wherein the oral health refers to periodontitis, gingivitis, halitosis, bad breath, or inflammation provoking conditions associated with oral care.

28. A process of preparing a probiotic formulation of claim 1, the process comprising:
a. making a blend of inert excipients selected from the group consisting of microcrystalline cellulose, maltodextrin, lactose, starch, FOS, Alpha-GOS, magnesium stearate, and silicon dioxide;
b. thawing and mixing the probiotic cultures with suitable portion of blend from (a);
c. mixing the blend of (b) with the blend of (a) under controlled processing parameters wherein the parameters comprise a temperature of 18 to 20° C. and a relative humidity of 20 to 25%; and
d. adding and mixing non-probiotic components selected from anticaking and lubricating agents in the blend of (c),
wherein the formulation generated is a low water activity blend consisting of oro-dispersible properties and maintains shelf-life of 24 to 36 months.

29. The process of claim 13, further comprising:
e. adding and mixing flavors to the blend of (d).
